(19) United States
(12) Patent Application Publication
(10) Pub. No.: US 2024/0277775 A1
(43) Pub. Date: Aug. 22, 2024

(54) PHARMACEUTICAL USE OF CORD BLOOD IMMUNOSUPPRESSIVE CELL

(71) Applicants: VIGENCELL INC., Seoul (KR); THE CATHOLIC UNIVERSITY OF KOREA INDUSTRY-ACADEMIC COOPERATION FOUNDATION, Seoul (KR)

(72) Inventors: Hyun Jung SOHN, Seoul (KR); Tai Gyu KIM, Seoul (KR); Su Eon KIM, Gyeonggi-do (KR); Soo Hyun JUNG, Seoul (KR); Hyun ll CHO, Gyeonggi-do (KR); Bang Geul LIM, Seoul (KR); Eun Ah KIM, Seoul (KR)

(21) Appl. No.: 18/569,023

(22) PCT Filed: Jun. 14, 2021
(86) PCT No.: PCT/KR2021/007420

§ 371 Date: Dec. 11, 2023

(51) Int. Cl. A61K 35/51; A61P 9/10; C12N 5/078

(52) U.S. Cl. CPC A61K 35/51; A61P 9/10; C12N 5/0634; C12N 2501/125; C12N 2501/22

(57) ABSTRACT

The present invention relates to the pharmaceutical use of a cord blood immunosuppressive cell and, more specifically, the present invention provides the use of a human-derived cord blood immunosuppressive cell, which is selected in accordance with analysis of function and phenotype and a classification standard, as a new cell therapy product for anti-inflammation, anti-fibrosis, and prevention or treatment of myocardial infarction.

### BACKGROUND

### 1. Field of the Invention

The present invention relates to the pharmaceutical use of a cord blood immunosuppressive cell having anti-inflammation, anti-fibrosis, and prevention or treatment potential for myocardial infarction.

### 2. Discussion of Related Art

Cord blood immunosuppressive cells are a collection of cord blood-derived differentiated cells with potent immunosuppressive effects. The cord blood immunosuppressive cell is a hematopoietic stem cell precursor that develops macrophages, dendritic cells, and granulocytes at various stages of hematopoietic differentiation, these cells are not present in healthy individuals, but the cord blood immunosuppressive cell is accumulated in peripheral blood, lymphatic organs, the spleen, cancer tissue, and the like in pathological conditions such as infection, inflammatory responses, cancer, and autoimmunity. Stimulatory factors such as SCH, VEGF, GM-CSF, G-CSF, and M-CSF, cytokines such as IFN-g, IL-1b, IL-6, IL-12, and IL-13, calcium binding proteins S100A8 and S100A9, complement component 3 (C3), cyclooxygenase-2, prostaglandin E2, and the like are factors that proliferate and activate the cord blood immunosuppressive cell.

Most of the cord blood immunosuppressive cells are known to exert immunosuppressive effects through direct cell-to-cell contact and perform immunosuppressive functions by secreting materials such as cytokines with short half-lives. Examples of agonists known to date include arginase I and inducible nitric oxide synthase (iNOS), reactive oxygen species (ROS), peroxynitrite, and the like, and among them, arginase I and iNOS are representative T cell suppressive materials, and directly suppress the proliferation of T cells, whereas ROS and peroxynitrite suppress the ability to recognize antigens through post-translational modification of T-cell receptors. Based on the studies on the functions and mechanisms of action of these cord blood immunosuppressive cells, efforts to develop a new therapeutic method for suppressing immune responses through their regulation have been recently accelerated.

In order to develop a cord blood immunosuppressive cell as a therapeutic agent, the cord blood immunosuppressive cell needs to be differentiated and expanded by in vitro culture, but it is difficult to mass-expand human-derived cord blood immunosuppressive cells. The biggest obstacle is that standardized and stable culture techniques for cord blood suppressor cells have not been established, and the classification criteria thereof are also ambiguous. Furthermore, because the amount of CD34-positive cells in blood is so small, there are many difficulties in not only securing, but also mass-producing CD34-positive cells.

### SUMMARY OF THE INVENTION

An object of the present invention is to provide a use of a human-derived cord blood immunosuppressive cell for anti-inflammation and anti-fibrosis, and for preventing or treating myocardial infarction.

Another object of the present invention is to provide a method for selecting a human-derived cord blood immunosuppressive cell with excellent immunosuppressive ability.

In order to achieve the objects, the present invention provides a composition for preventing or treating inflammation, fibrosis, or myocardial infarction, including a cord blood immunosuppressive cell.

The present invention also provides a method for treating inflammation, fibrosis, or myocardial infarction, the method including: administering a therapeutically effective amount of cord blood immunosuppressive cells to a subject in need thereof.

The present invention also provides a method for selecting a cord blood-derived immunosuppressive cell with excellent immunosuppressive ability, the method including: classifying cord blood immunosuppressive cells having a phenotype of CD33+CD11b+ and selecting a cord blood immunosuppressive cell expressing a phenotype of CD14+ using CD15 positive cells as a positive control among the cord blood immunosuppressive cells;
selecting a cord blood immunosuppressive cell having an HLA-DR^{LOW} phenotype among cord blood immunosuppressive cells having a cell phenotype of CD11b+CD33+CD14+ compared to HLA-DR-positive cells; and
co-culturing peripheral blood mononuclear cells and cord blood immunosuppressive cells under magnetic beads at a concentration of 0.125 to 2 μl/mL to confirm the proliferation ability of T cells, and
co-culturing peripheral blood mononuclear cells and an immunosuppressant, as a control, at a concentration of 0.05 to 320 ng/mL under magnetic beads at a concentration of 0.125 to 2 μl/mL to confirm the proliferation ability of T cells and compare the T cell proliferation ability of the cord blood immunosuppressive cells with that of the control,
wherein the cord blood immunosuppressive cell is induced by culturing CD34-positive cells isolated from human cord blood in a cell culture medium including a cytokine combination of GM-CSF and SCF for 2 to 7 weeks.

The present invention has an effect of providing a human-derived cord blood immunosuppressive cell with excellent immunosuppressive ability according to the analysis and classification criteria of function and phenotype.

Human-derived cord blood immunosuppressive cells selected according to the above functional and phenotypic analysis and classification criteria of the present invention can be used as a new cell therapeutic agent for preventing or treating inflammation, fibrosis, or myocardial infarction through an increase in differentiation and migration of an inflammatory suppressor cell (M2 macrophage), a decrease in differentiation and migration of an inflammation-inducing cell (M1 macrophage), an improvement in cardiac function (end-systolic volume (ESV), fraction shortening (FS), and ejection fraction (EF)), a decrease in myocardial infarction size or an increase in migration to the heart during myocardial infarction.

### BRIEF DESCRIPTION OF THE DRAWINGS

FIG. 1 illustrates the results of analyzing the immunosuppressive ability of cord blood immunosuppressive cells (CBICs) according to the concentration of magnetic beads (Dynabeads) for T cell stimulation.

FIG. 2 illustrates the results of analyzing the immunosuppressive ability of cord blood immunosuppressive cells (CBICs) according to the concentration of an immunosuppressant.

FIG. 3 illustrates the results of selecting cord blood immunosuppressive cells (CBICs) according to phenotype using anti-CD33/CD11b/CD14 antibodies.

FIG. 4 illustrates the results of selecting cord blood immunosuppressive cells (CBICs) according to phenotype using a CD15 positive control.

FIG. 5 illustrates the results of selecting cord blood immunosuppressive cells (CBICs) according to the HLA-DR phenotype using a positive control expressing HLA-DR (genetically modified K562 cell line and dendritic cells).

FIG. 6 illustrates the results of confirming the phenotype and quality of cord blood immunosuppressive cells by lot.

FIG. 7 illustrates the results of confirming the expression of immunosuppressive proteins iNOS2, arginase I, and IDO in cord blood immunosuppressive cells.

FIG. 8 illustrates the results of confirming the in vitro immunosuppressive ability of cord blood immunosuppressive cells.

FIG. 9 illustrates the effects in which cord blood immunosuppressive cells (CBICs) change the sizes of myocardial infarction sites (anti-fibrosis:infart size) and improve the cardiac function indices (end-systolic volume (ESV), fraction shortening (FS), and left ventricular ejection fraction (LVEF)).

FIG. 10 illustrates the effects of cord blood immunosuppressive cells (CBIC) on the differentiation and migration of a proinflammatory cell (M1 macrophage) and an inflammatory suppressor cell (M2 macrophage) in an LAD ligation model.

FIG. 11 illustrates the effect of cord blood immunosuppressive cells (CBIC) on changes in sizes of myocardial infarction sites (anti-fibrosis:infart size) and improvements in cardiac function indices (ESV, FS, and EF) in a dose-dependent manner in the LAD ligation model.

FIG. 12 illustrates dose- and frequency-dependent effects on the viability of cord blood immunosuppressive cells in the LAD ligation model.

FIG. 13 illustrates the migration results of cord blood immunosuppressive cells (CBIC) to the heart in a myocardial infarction mouse model.

### DETAILED DESCRIPTION OF EXEMPLARY EMBODIMENTS

Hereinafter, the configuration of the present invention will be described in detail.

The present invention relates to a composition for preventing or treating inflammation, fibrosis, or myocardial infarction, including a cord blood immunosuppressive cell.

The cord blood immunosuppressive cell of the present invention may be induced by culturing CD34-positive cells isolated from human cord blood in a cell culture medium including a cytokine combination of GM-CSF and SCF for a predetermined time.

The CD34-positive cells may be isolated by a conventional isolation method, and may be isolated using, for example, a human anti-CD34 antibody.

The cord blood immunosuppressive cells of the present invention may be expanded and differentiated by culturing the CD34-positive cells in a cell culture medium containing GM-CSF and SCF for 2 to 7 weeks, more specifically 3 to 6 weeks. More preferably, the period may be 3 to 6 weeks, but is not limited thereto. According to an embodiment, when cultured for 3 to 6 weeks, the CD34-positive cells may be induced to differentiate into cord blood immunosuppressive cells with a CD11b⁺ CD33⁺ expression of 30% to 95%.

The cell culture medium may be a safe medium for animal cell culture. Examples of the safe medium include Dulbecco's Modified Eagle's Medium (DMEM), Minimal essential Medium (MEM), Basal Medium Eagle (BME), RPMI1640, F-10, F-12, αMinimal essential Medium (αMEM), Glasgow's Minimal essential Medium (GMEM), Iscove's Modified Dulbecco's Medium, and the like, but are not limited thereto.

The GM-CSF and SCF may be added to the cell culture medium at a concentration ratio of 1:0.8 to 0.3.

Preferably, the GM-CSF may be added to the cell culture medium at a concentration of 50 ng/mL to 200 ng/mL. The SCF may be added to the cell culture medium at a concentration of 10 ng/mL to 100 ng/mL. Within the above range, the proliferation of CD34⁺ cells may be relatively increased. According to an embodiment, when CD34-positive cells are cultured under G-CSF/SCF for 3 weeks, the cells proliferate about 600-fold, but may proliferate 1000- to 3000-fold under GM-CSF/SCF.

CD34-positive cells may differentiate into cord blood immunosuppressive cells under an aeration amount of 5 to 15% carbon dioxide at 35 to 37° C. in a CO₂ incubator, but are not particularly limited thereto.

The cord blood immunosuppressive cells induced to differentiate and proliferate under the above conditions may proliferate 1000- to 3000-fold based on the number of CD34⁺ cells at the initial culture stage.

As used herein, the term “cord blood derived immunosuppressive cell” refers to immature myeloid cells, which are present in an immature state because granulocytes and the like do not completely differentiate in tumors, autoimmune diseases, and infections, and the cord blood immunosuppressive cells are known to increase not only in cancer patients but also in acute inflammatory diseases, trauma, sepsis, and parasitic and fungal infections. The function of cord blood immunosuppressive cells is to effectively suppress activated T cells. For the mechanism by which cord blood immunosuppressive cells regulate T cells, it is known that enzymes such as nitric oxide synthase, reactive oxygen species (ROS), and arginase suppress the activity of T cells by maximizing the metabolism of L-arginine, which is an essential amino acid. Therefore, the cord blood immunosuppressive cell of the present invention, which is induced to differentiate from the CD34-positive cells isolated from cord blood may be a monocytic cord blood immunosuppressive cell expressing CD11b+, CD33+, CD14+, CD15- and a cell phenotype including HLA-DR^{LOW} within a set range, that is, 30% or less. As used herein, the cord blood immunosuppressive cell of the HLA-DR^{LOW} phenotype refers to a cell exhibiting an HLA-DR expression level of 30% or less compared to the HLA-DR expression level of a HLA-DR-positive cell. The cord blood immunosuppressive cell may also include the expression of PDL-1, CCR2, CCR5, CD62L, CXCR4 and ICAM-1 as cell surface markers.

According to an embodiment of the present invention, when the cell surface was stained after culturing CD34-positive cells isolated from cord blood under GM-CSF and SCF for 6 weeks, HLA-ABC, HLA-DR, and CD45 were expressed at 70%, 30% or less, and 90% or more, respectively, and 10% expression of CD83 and CD80 was observed only in a cord blood immunosuppressive cell induced to differentiate under the GM-CSF/SCF combination of the present invention compared to a cord blood immunosuppressive cell induced to differentiate under the G-CSF/SCF combination. CD86 was expressed at about 40% in a cord blood immunosuppressive cell by the GM-CSF/SCF combination, showing the low expression pattern of co-stimulatory molecules. In addition, CD40 was expressed at 40%, and lymphocyte markers CD1d, CD3, and B220 were expressed at less than 5%. PDL-1, which is known to suppress the proliferation and activation of T cells, was expressed at about 30% only in cells cultured by the GM-CSF/SCF combination. CD13 is a transmembrane glycoprotein expressed in myeloid precursors, myeloperoxidase (MPO) is a protein in the azurophilic granules of myeloid cells, both of which are proteins expressed in cord blood immunosuppressive cells. Cord blood immunosuppressive cells induced by the combination of GM-CSF/SCF had significantly increased CD13 expression than cord blood immunosuppressive cells induced by the combination of G-CSF/SCF. MPO was expressed at 90% or more in both cord blood immunosuppressive cells induced by two different combinations.

Furthermore, cord blood immunosuppressive cells induced by the combination of GM-CSF/SCF have increased expression of a T cell inhibitory substances including arginase I, inducible nitric oxide synthase (iNOS), and indoleamine 2,3-dioxygenase (IDO) compared to cord blood immunosuppressive cells induced by the combination of G-CSF/SCF and human peripheral blood-derived dendritic cells.

Cord blood immunosuppressive cells induced by the combination of GM-CSF/SCF strongly reduce the secretion of IFN-γ by antigen-specific T cell immune responses by significantly suppressing the proliferation of allogeneic CD4 T cells. It was observed that the secretion of IL-10 was significantly increased when cord blood immunosuppressive cells induced by the combination of GM-CSF/SCF were stimulated with a CD40 antibody, and VEGF and TGF-β are highly secreted regardless of whether they are stimulated with an antibody. Further, it is known that when CD4 T cells are stimulated in vitro by cord blood immunosuppressive cells, Treg cells expressing FoxP3 are increased, and when CD4 T cells are stimulated by cord blood immunosuppressive cells induced by the combination of GM-CSF/SCF, FoxP3 expression is confirmed, but IL-17, which is an inflammatory cytokine, is not secreted.

The cord blood immunosuppressive cell of the present invention, according to an embodiment, may be used as a new cell therapeutic agent for preventing or treating inflammation, fibrosis, or myocardial infarction through an increase in differentiation and migration of an inflammatory suppressor cell (M2 macrophage), a decrease in differentiation and migration of a proinflammatory cell (M1 macrophage), an improvement in cardiac function (end-systolic volume (ESV), fraction shortening (FS), and ejection fraction (EF)), a decrease in myocardial infarction size or an increase in migration to the heart during myocardial infarction.

The composition for preventing or treating inflammation, fibrosis or myocardial infarction of the present invention may further include a pharmaceutically acceptable carrier.

The pharmaceutically acceptable carrier includes a carrier and a vehicle typically used in the medical field, and specific examples thereof include an ion exchange resin, alumina, aluminum stearate, lecithin, a serum protein (for example, a human serum albumin), a buffer substance (for example, various phosphates, glycine, sorbic acid, potassium sorbate, partial glyceride mixture of saturated vegetable fatty acid), water, a salt or electrolyte (for example, protamine sulfate, dissodium hydrogen phosphate, potassium hydrogen phosphate, sodium chloride, and zinc salts), colloidal silica, magnesium trisilicate, polyvinylpyrrolidone, cellulose-based substrate, polyethylene glycol, sodium carboxymethylcellulose, polyarylate, wax, polyethylene glycol, wool, or the like, but are not limited thereto.

In addition, the pharmaceutical composition of the present invention may additionally include a lubricant, a wetting agent, an emulsifier, a suspending agent, a preservative, or the like, in addition to the aforementioned ingredients.

As an aspect, the composition according to the present invention may be prepared as an aqueous solution for parenteral administration, and preferably, a buffer solution such as Hank's solution, Ringer's solution or physically buffered saline may be used. A substrate capable of increasing the viscosity of the suspension, such as sodium carboxymethyl cellulose, sorbitol or dextran may be added to an aqueous injection suspension.

The composition of the present invention may be administered systemically or topically, may be administered, for example, by oral, parenteral, for example, suppository, transdermal, intravenous, intraperitoneal, intramuscular, intralesional, intranasal, and intraspinal administration, and may be administered using an implantable device for sustained or continuous or repeated release. Administration may be carried out once or several times a day within a desired range, and the administration period is not also particularly limited.

Furthermore, formulations suitable for such administration may be prepared by a known technique. For example, during the oral administration, the composition of the present invention may be administered while being mixed with an inert diluent or an edible carrier, sealed in a hard or soft gelatin capsule, or compressed into a tablet. For oral administration, the active compound may be mixed with an excipient and used in the form of an ingestible tablet, a buccal tablet, a troche, a capsule, an elixir, a suspension, a syrup, a wafer, and the like. Various dosage forms for injection and parenteral administration may be prepared by techniques known in the art or commonly used techniques. Intravenous injection, subcutaneous injection, intramuscular injection, intraperitoneal injection, transdermal injection and the like may be used for dosage form administration.

The dosage of the composition of the present invention for a patient varies depending on many factors including the patient's height, body surface area, and age, the particular compound administered, sex, time and route of administration, general health, and other drugs administered concurrently. Typically, cord blood immunosuppressive cells may be administered at typically about 10⁹ to 10¹⁰ cells per m² of body surface area per administration. Therefore, although it is appropriate to administer about 2×10¹⁰ cells based on a general adult (about 60 kg), the above dosage may vary depending on the patient's various conditions and the types and amounts of drugs co-administered as described above. Therefore, the cord blood immunosuppressive cell of the present invention, which is pharmaceutically active, may be administered in an amount of 10⁶ to 10¹⁰ cells/kg (body weight), and dosages below or above the above exemplary ranges are also contemplated, particularly considering the above factors. When the method of administration is continuous infusion, the dosage needs to be in a range of 10³ to 10⁹ cell units per kilogram of body weight per minute.

The present invention also provides a method for treating inflammation, fibrosis, or myocardial infarction, the method including: administering a therapeutically effective amount of a cord blood immunosuppressive cell to a subject in need thereof.

As used herein, “therapeutically effective amount” refers to an amount capable of ameliorating, alleviating or treating inflammation, fibrosis or myocardial infarction.

The subject may include a human, a dog, a chicken, a pig, a cow, a sheep, a guinea pig, a monkey, a mouse, a rat, and the like.

The present invention also relates to a method for selecting a cord blood-derived immunosuppressive cell with excellent immunosuppressive ability, the method including: classifying cord blood immunosuppressive cells having a phenotype of CD33+CD11b+ and selecting a cord blood immunosuppressive cell expressing a phenotype of CD14+ using CD15 positive cells as a positive control among the cord blood immunosuppressive cells;
selecting a cord blood immunosuppressive cell having an HLA-DR^{LOW} phenotype among cord blood immunosuppressive cells having a cell phenotype of CD11b+CD33+CD14+ compared to HLA-DR-positive cells; and
co-culturing peripheral blood mononuclear cells and cord blood immunosuppressive cells under magnetic beads at a concentration of 0.125 to 2 μl/mL to confirm the proliferation ability of T cells, and
co-culturing peripheral blood mononuclear cells and an immunosuppressant, as a control, at a concentration of 0.05 to 320 ng/mL under magnetic beads at a concentration of 0.125 to 2 μl/mL to confirm the proliferation ability of T cells and compare the T cell proliferation ability of the cord blood immunosuppressive cells with that of the control,
wherein the cord blood immunosuppressive cell is induced by culturing CD34-positive cells isolated from human cord blood in a cell culture medium including a cytokine combination of GM-CSF and SCF for 2 to 7 weeks.

The method for selecting a cord blood-derived cord blood immunosuppressive cell with excellent immunosuppressive ability of the present invention is characterized by selecting a cord blood-derived cord blood immunosuppressive cell with excellent immunosuppressive ability by confirming phenotype selection criteria, that is, criteria of selecting a cell phenotype of CD11b+CD33+CD14+ and an HLA-DR^{LOW} phenotype, and selection criteria for confirming the proliferation ability of T cells, that is, magnetic beads by which the proliferation ability of T cells is easily confirmed and the concentration range of an immunosuppressant, and co-culturing with peripheral blood mononuclear cells using them as a control to compare the proliferation abilities of T cells.

Therefore, the first step is to confirm the cell phenotype, that is, a cell phenotype of CD11b+CD33+CD14+ and the HLA-DR^{LOW} phenotype.

For the CD15-positive cells, granulocytes; and a genetically modified cell line so as to express a CD15 gene, for example, a genetically modified cell line using CD15DNA, a lentivirus including the CD15 gene, CD15IVT mRNA, and the like may be used.

For the HLA-DR-positive cells, dendritic cells; monocytes; and a genetically modified cell line so as to express an HLA-DR gene, for example, a genetically modified K562 cell line using HLA-DR DNA, a lentivirus including the HLA-DR gene, HLA-DR IVT mRNA, and the like may be used.

The HLA-DR^{LOW} phenotype refers to a cord blood immunosuppressive cell exhibiting an expression level of 30% or less compared to the HLA-DR expression level of a HLA-DR-positive cell.

The second step is a step of determining the concentration range of magnetic beads and immunosuppressants that can easily confirm the proliferation ability of T cells, as a control; determining the proliferative ability of T cells by co-culturing umbilical cord blood immunosuppressive cells whose cell phenotypes were confirmed, and peripheral blood mononuclear cells; and selecting umbilical cord blood immunosuppressive cells with excellent T cell proliferation ability by comparing them with the control.

Magnetic beads co-cultured with peripheral blood mononuclear cells may be used at a concentration of 0.125 to 2 μl/mL. When a concentration within the above range was used, the analysis of immunosuppressive ability was easiest during the induction of T cell proliferation.

When T cell proliferation is induced by co-culturing peripheral blood mononuclear cells and magnetic beads, the immunosuppressant used as a control may be used at a concentration of 0.05 to 320 ng/mL. When a concentration within the above range was used, the analysis of immunosuppressive ability was easiest during the induction of T cell proliferation.

The immunosuppressant may be used in rapamycin, cyclosporin A, tacrolimus, mycophenolic acid, azathioprine, Bredinin, silorimus, everolimus, or the like.

The peripheral blood mononuclear cells and cord blood immunosuppressive cells may be co-cultured at a cell number ratio of 1:0.25 to 1.

Hereinafter, the present invention will be described in detail with reference to Examples according to the present invention. However, the present invention is not limited to the following Examples.

### <Example 1> Induction and Proliferation of Cord Blood Immunosuppressive Cells CBICs

After CD34+ cells were isolated from cord blood from different individuals, 1×10⁵ of the cells began to be cultured using an IMDM medium with the cytokine combination of GM-CSF (100 ng/mL)/SCF (50 ng/mL) or G-CSF (100 ng/mL)/SCF (50 ng/mL) in a 48-well plate, and the expansion of CD34+ cells was induced.

As a result, in the combination of GM-CSF/SCF, the cells were expanded 10-fold or more at week 1, 100-fold or more at week 2, and 1,000-fold or more at week 3, whereas in the combination of G-CSF/SCF, the cells were expanded 600-fold at week 3. Therefore, it could be seen that the combination of GM-CSF (100 ng/mL)/SCF (50 ng/mL) more efficiently expanded CD34+ cells.

Next, after CD34+ cells were isolated from cord blood, the cells cultured with GM-CSF (100 ng/mL)/SCF (50 ng/mL) or G-CSF (100 ng/mL)/SCF (50 ng/mL) for 6 weeks, and then analyzed by a flow cytometer. As a result of confirming the expression of CD11b+CD33+ after gating Lin-cells, it was confirmed that, in GM-CSF/SCF, CD11b+CD33+ were expressed at 30% or more at week 3 and a cord blood immunosuppressive cell (CBIC) group was expressed at about 90% through long-term culture for 6 weeks. In contrast, in G-CSF/SCF, CD11b+CD33+ was expressed at about 15% at week 3, and then gradually decreased cell groups were observed. It was confirmed that the combination of GM-CSF/SCF highly efficiently induced the differentiation of cord blood immunosuppressive cells (CBICs).

Next, the expression of immunosuppressive proteins was measured in cord blood immunosuppressive cells (CBIC) induced to differentiate from cord blood-derived CD34+ cells.

For this purpose, as a result of comparing the expression of iNOS2, arginase I, and IDO in cord blood immunosuppressive cells (CBICs) cultured for 6 weeks, it was observed that iNOS2 and IDO were expressed more significantly in GM-CSF/SCF than in the combination of G-CSF/SCF. Although arginase I was also expressed higher in the combination of GM-CSF/SCF than in the combination of G-CSF/SCF, the difference between both combinations did not show significance.

### <Example 2> Criteria for Classifying Cord Blood Immunosuppressive Cells According to Phenotype and Function

The criteria for classifying cord blood immunosuppressive cells according to phenotype and function have not yet been established. Therefore, normal cord blood immunosuppressive cells (CBICs) according to their function and phenotype were classified using a clear control, and by using this, cells were clearly defined and the functions thereof were confirmed.

(1) Establishment of Immunosuppressive Ability Analysis Criteria for Selecting Cord Blood Immunosuppressive Cells (CBICs) with Normal Function According to the Difference in Concentration of Magnetic Beads (Dynabeads) for T Cell Stimulation

It was difficult to fully observe the immunosuppressive ability of cord blood immunosuppressive cells produced when T cells were stimulated using 2 μl of Dynabeads. Therefore, in order to establish a suitable concentration for Dynabeads, it was determined, using Dynabeads at each concentration, whether there was a change in the suppression of proliferation of CD4 and CD8 T cells. Specifically, PBMCs labeled with carboxyfluorescein succinimidyl ester (CFSE) were co-cultured with CBICs at a ratio of 1:1, 1:0.5, and 1:0.25 (PBMCs:CBICs) for 6 days using 2 μl, 0.5 μl, 0.25 μl, and 0.125 μl of Dynabeads, which are magnetic beads that stimulate T cells. Next, after the cell surface was stained using anti-CD3, CD4, and CD8 antibodies, the proliferation ability of T cells was confirmed.

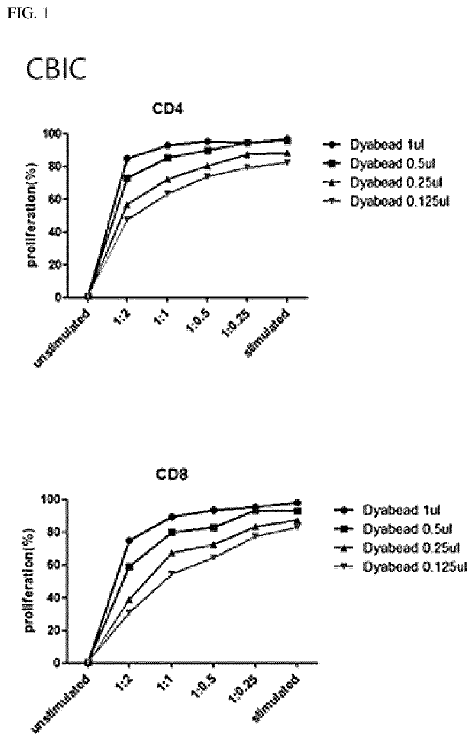

As illustrated in FIG. 1, it was confirmed that the immunosuppressive ability analysis was easiest when T cells were proliferated with 0.125 μl of Dynabeads.

(2) Establishment of Immunosuppressive Ability Analysis Criteria for Selecting Cord Blood Immunosuppressive Cells (CBICs) with Normal Function According to the Difference in Concentration of Immunosuppressant

It was difficult to fully observe the immunosuppressive ability of rapamycin and CsA used as controls when T cells were stimulated using 2 μl of Dynabeads. In order to establish a suitable concentration for Dynabeads, it was determined, using Dynabeads at each concentration, whether there was a change in the suppression of proliferation of CD4 and CD8 T cells. Specifically, in order to establish the criteria for analyzing the immunosuppressive ability of CBICs, rapamycin and cyclosporin A (CsA) as controls were serially diluted to each concentration and used. PBMCs labeled with CFSE were co-cultured with CBICs at a ratio of 1:1, 1:0.5, and 1:0.25 (PBMCs:CBICs) for 6 days using 2 μl, 0.5 μl, 0.25 μl, and 0.125 μl of Dynabeads, which are magnetic beads that stimulate T cells. Next, after the cell surface was stained using anti-CD3, CD4, and CD8 antibodies, the proliferation ability of T cells was confirmed.

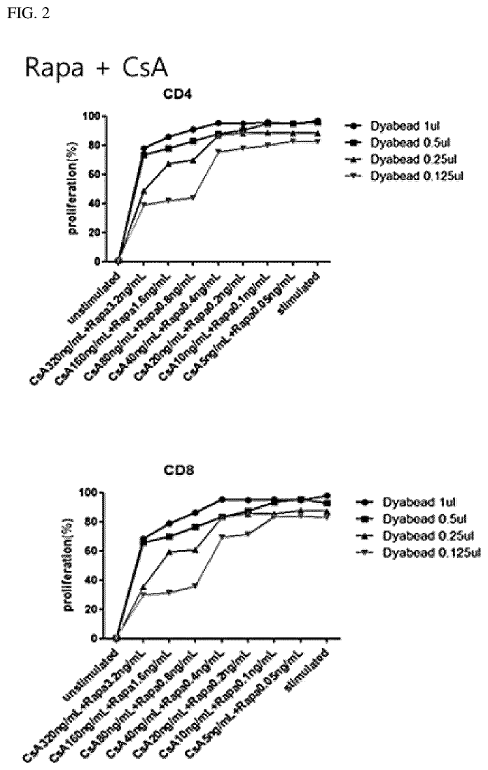

As illustrated in FIG. 2, it was confirmed that the immunosuppressive ability analysis was easiest when T cells were proliferated with 0.125 μl of Dynabeads.

### (3) Establishment of Phenotypic Analysis Criteria for Selecting Normal Cord Blood Immunosuppressive Cells (CBICs) According to Phenotype

In order to analyze the phenotype of CBICs cultured for 6 weeks, the cell surface was stained using anti-CD33/CD11b/CD14 antibodies and then analyzed using a flow cytometer. In this case, unstained CBICs were used as a control.

As a result, the phenotype of cord blood immunosuppressive cells was confirmed and detailed analysis criteria were established. Cord blood immunosuppressive cells are broadly classified into two types, granulocyte-myeloid immunosuppressive cells (G-MICs) or monocyte-myeloid immunosuppressive cells (M-MICs). It is known that granulocyte-myeloid immunosuppressive cells exhibit a phenotype of CD33+CD11b+CD15+CD14-, and monocyte-myeloid immunosuppressive cells exhibit a phenotype of CD33+CD11b+CD15-CD14+. CBICs commonly exhibit a phenotype of CD33+CD11b+ and may be classified into granulocyte-myeloid immunosuppressive cells and monocyte-myeloid immunosuppressive cells by expression of CD15 and CD14. Monocyte-myeloid immunosuppressive cells are generally known to have stronger immunosuppressive reactions than granulocyte-myeloid immunosuppressive cells.

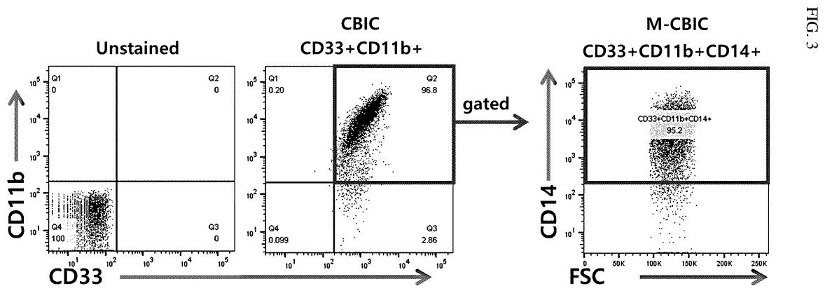

As illustrated in FIG. 3, it was confirmed that the cord blood immunosuppressive cells induced in the example exhibited CD33+CD11b+, which is a common phenotype of myeloid immunosuppressive cells, and the detailed phenotype is CD33+CD11b+CD14+, which is similar to the phenotype of monocyte-myeloid immunosuppressive cells.

(4) Establishment of Phenotypic Analysis Criteria which Classify Whether Other Cells are Mixed in Cord Blood Immunosuppressive Cells (CBICs) Using a Positive Control Group

When a granulocyte, which is a CD15 positive control, a CD15+ genetically modified cell line, a monocyte, which is a CD14-positive control, and a CD14+ genetically modified cell line were classified according to the criteria, the phenotype of the CBIC was identified as CD33+CD11b+CD14+/CD15-. Furthermore, it is known that there are no double-positive cells of CD14 and CD15. Therefore, in order to analyze the phenotype of CBICs cultured for 6 weeks, the cell surface was stained with anti-CD33/CD11b/CD15 antibodies and then analyzed using a flow cytometer. In this case, unstained CBICs were used as a control.

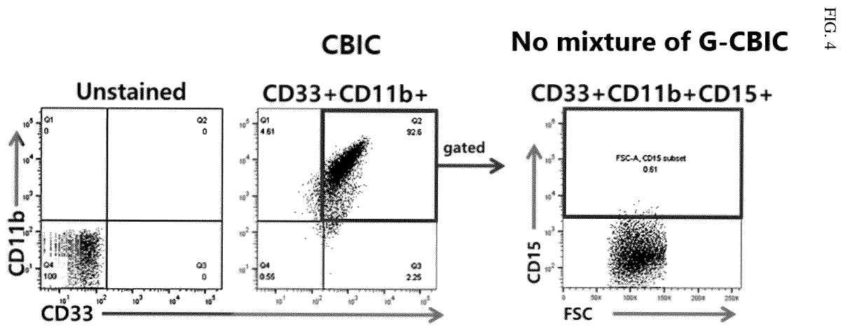

As shown in FIG. 4, it can be confirmed that CD15+ is not mixed because CD33+CD11b+CD14+ cells are at a level of 93.9 to 99.6%.

(5) Establishment of HLA-DR Phenotypic Analysis Criteria for Selecting Normal Cord Blood Immunosuppressive Cells (CBIC) Separately from Monocytes Using an HLA-DR Positive Control

Since phenotypic studies of cord blood immunosuppressive cells have not yet been perfected, it has been difficult to completely classify cells. Granulocyte-myeloid immunosuppressive cells are phenotypically similar to neutrophils, and monocyte-myeloid immunosuppressive cells are phenotypically similar to monocytes.

In the present invention, the phenotypes of monocyte-myeloid immunosuppressive cells and monocytes were classified into HLA-DR negative phenotypes using dendritic cells highly expressing HLA-DR and a K562 cell line, thereby finally producing cord blood immunosuppressive cells that were phenotypically different from monocytes.

Specifically, the surface of CBIC cells cultured for 6 weeks was stained using anti-CD33/CD11b/HLA-DR antibodies and then analyzed using a flow cytometer. Dendritic cells expressing HLA-DR and the K562 cell line were used as controls after staining the cell surface using an anti-HLA-DR antibody.

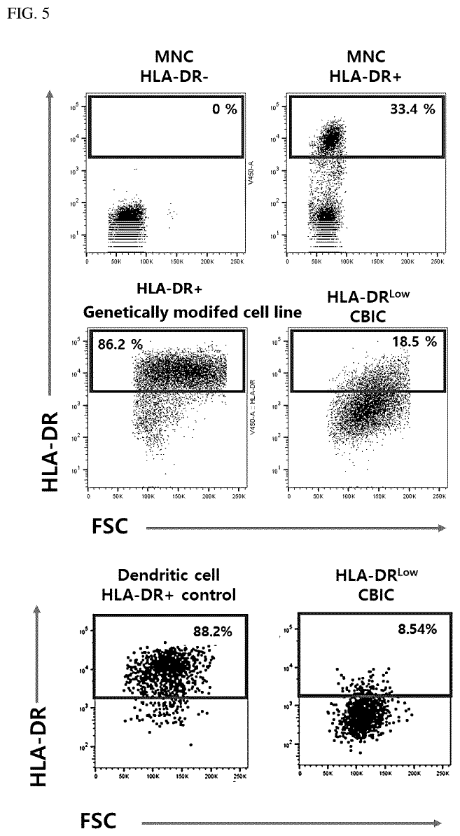

As illustrated in FIG. 5, an HLA-DR phenotype lower than the control could be confirmed, which is considered suitable for HLA-DR Low, which is a phenotype of M-CBIC.

### (6) Results of Confirming Phenotype and Quality of Cord Blood Immunosuppressive Cells by Lot

In order to analyze the phenotype of CBICs cultured for 6 weeks, the cell surface was stained using anti-CD33/CD11b/CD14 antibodies and then analyzed using a flow cytometer. In this case, unstained CBICs were used as a control.

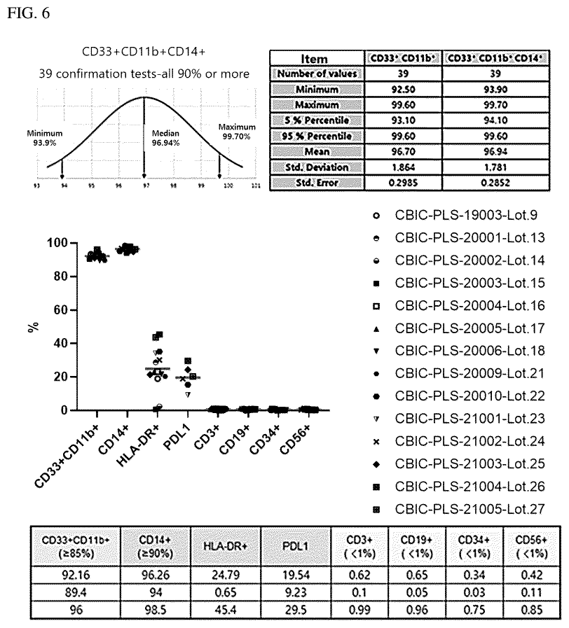

As illustrated in FIG. 6, the CD33+CD11b+CD14+ expression rate was 93.9% at minimum, 99.70% at maximum, and 96.94% at median (on average), all 39 measurements were reproduced above the reference value of 90%, and the quality was repeatedly maintained.

(7) Confirmation of Expression of Immunosuppressive Proteins iNOS2, Arginase 1, and IDO in Cord Blood Immunosuppressive Cells

In order to analyze the intracellular materials of CBICs cultured for 6 weeks, the cells were fixed using Lyse/Fix buffer at 37° C. for 10 minutes, then Perm buffer was added thereto and the resulting mixture was left to stand on ice for 30 minutes to induce perforations in the outer wall of the cell. Thereafter, anti-iNOS2/arginase/IDO antibodies were added, intracellular staining was performed, and then analysis was performed using a flow cytometer. In this case, CBICs, to which an intracellular staining antibody had not been added, were used as a control.

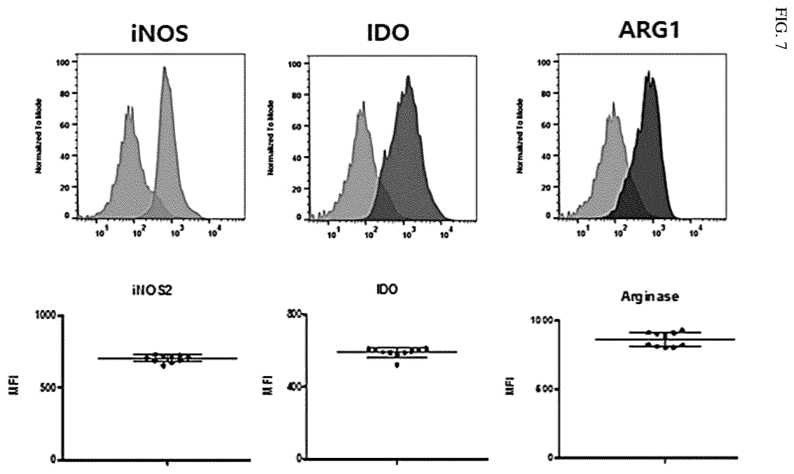

As illustrated in FIG. 7, cord blood immunosuppressive cells also expressed arginase I, inducible nitric oxide synthase (iNOS), and indoleamine 2,3-deoxygenase (IDO), which are immunosuppressive materials, without any difference between lots.

### (8) Confirmation of In Vitro Immunosuppressive Ability of Cord Blood Immunosuppressive Cells

In order to analyze the function of suppressing the proliferation of human helper T cells through cord blood immunosuppressive cells, CD4 T cells (1×10⁵) labeled with CFSE (using the concentration of 5 μM) from normal adults were cultured in a 96-well plate for 6 days using Dynabeads.

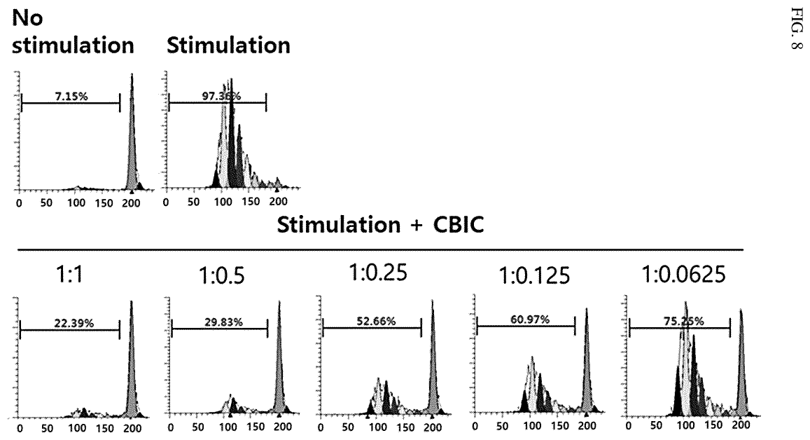

As illustrated in FIG. 8, the proliferation of human helper T cells occurred through Dynabeads, whereas the proliferation of helper T cells was suppressed in a group where cord blood immunosuppressive cells (1×10⁵) were cultured.

### <Example 3> Effect of Cord Blood Immunosuppressive Cells (CBICs) on Changes in Size of Myocardial Infarction Site (Anti-Fibrosis) and Cardiac Function Indices (ESV, FS, and EF)

A LAD ligation model was established to test the effect of cord blood immunosuppressive cells (CBICs) on anti-fibrosis of the myocardial infarction site and cardiac function indices (ESV, FS, and EF), and after cord blood immunosuppressive cells were administered, the size of the myocardial infarction site was confirmed through MT staining and compared to a control (PBS) to which cord blood immunosuppressive cells were not administered. Further, heart tissue was fixed with 4% paraformaldehyde, and then paraffin-fixed, sectioned into a size of 4 μm, and stained with Masson-Trichrome. After scanning with a slide scanner, the sections were analyzed by Image J.

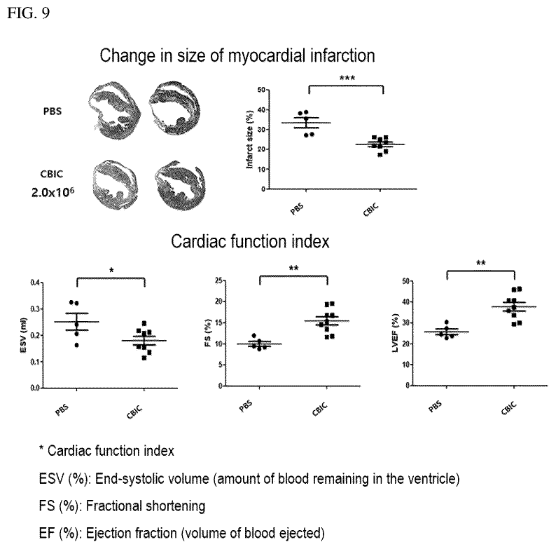

As illustrated in FIG. 9, it was confirmed that, in a myocardial infarction mouse model, the size of a myocardial infarction site was reduced in mice to which cord blood immunosuppressive cells were administered compared to a control to which cord blood immunosuppressive cells were not administered.

Next, a LAD ligation model was established to confirm cardiac function, and after cord blood immunosuppressive cells were administered, cardiac function was confirmed by ultrasound compared to a control (PBS) to which cord blood immunosuppressive cells were not administered. MRI was performed using BioSpec 47/40 (Bruker, Etlingen, Germany), and dual electrocardiogram (ECG) and respiratory gating were performed. ECG signals were obtained from needle electrodes in order to obtain MR images. Signals were transmitted and received using a quadrature birdcage RF resonator (Bruker) with an internal diameter of 72 mm, and ECG signals were obtained using R-waves from needle electrodes fixed on the forepaws and hindpaws in order to obtain MR images. Imaging parameters: FOV=60×60 mm², matrix size=256×256, slice thickness=1.5 mm, number of slices=1, TR=8 ms, TE=2.8 ms, flip angle=30°, average number=6, total scan time=3 min 16s. Ejection fraction (EF) and fraction shortening (FS) were measured by M-mode tracking at the papillary muscle level.

As illustrated in FIG. 9, it was confirmed that, in a myocardial infarction mouse model, cardiac function indices were improved in mice to which cord blood immunosuppressive cells were administered compared to a control to which cord blood immunosuppressive cells were not administered.

### <Example 4> Effect of Cord Blood Immunosuppressive Cells (CBICs) on Inflammatory Suppressor Cells and Proinflammatory Cells

The effect of cord blood immunosuppressive cells (CBICs) on inflammatory suppressor cells and proinflammatory cells was confirmed. For this purpose, a LAD ligation model was established, and after cord blood immunosuppressive cells were administered, M1 and M2 macrophages, which are immune effect markers, were confirmed by immunohistochemistry (IHC) compared to a control (PBS) to which cord blood immunosuppressive cells were not administered. Further, paraffin-embedded sections were deparaffinized and rehydrated, reacted with primary antibodies CD68, iNOS, and CD206 at 4° C. overnight, and then reacted with secondary antibodies at RT for 1 hour. The resulting product was confirmed under a fluorescence microscope after DAPI staining (LSM 510 Meta; Zeiss, Jena, Germany).

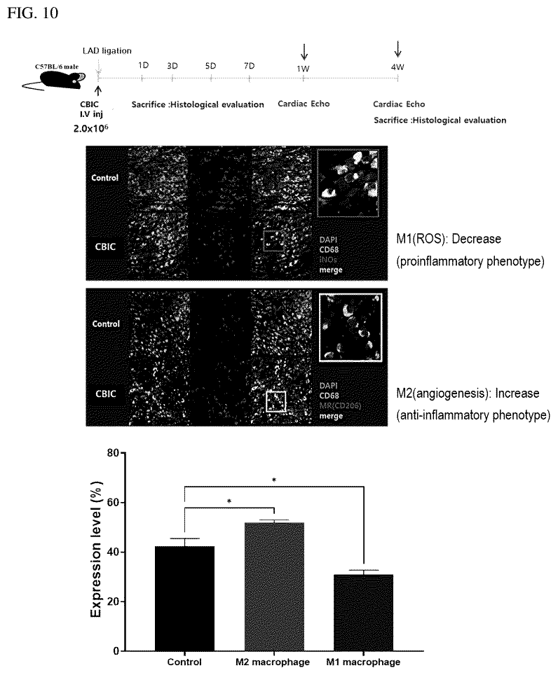

As illustrated in FIG. 10, in a myocardial infarction mouse model, a decrease in M1 (CD68+iNOS) macrophages and an increase in M2 (CD68+CD206) macrophages were confirmed in mice to which cord blood immunosuppressive cells were administered compared to a control to which cord blood immunosuppressive cells were not administered.

### <Example 5> Effect on Changes in Size of Myocardial Infarction Site (Anti-Fibrosis) and Cardiac Function Indices (ESV, FS, and EF) by Administration of Cord Blood Immunosuppressive Cells (CBICs) to Subject

By administering cord blood immunosuppressive cells (CBICs) to subjects at different concentrations, the effect on changes in size of a myocardial infarction site (anti-fibrosis) and cardiac function indices (ESV, FS, and EF) was confirmed. For this purpose, a LAD ligation model was established, and after cord blood immunosuppressive cells were administered, the size of the myocardial infarction site was confirmed by the MT stanning of the myocardial infarction site compared to a control (PBS) to which cord blood immunosuppressive cells were not administered. Heart tissue was fixed with 4% paraformaldehyde, and then paraffin-fixed, sectioned into a size of 4 μm, and stained with Masson-Trichrome. After scanning with a slide scanner, the sections were analyzed by Image J.

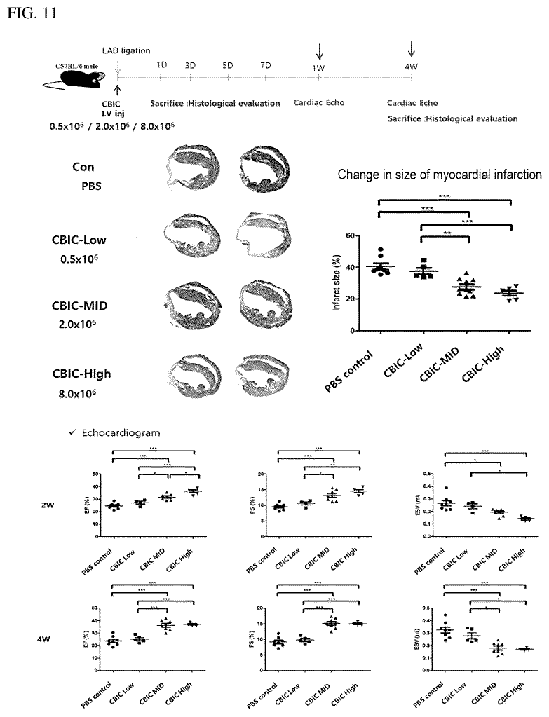

As illustrated in FIG. 11, it was confirmed that, in a myocardial infarction mouse model, the size of a myocardial infarction site was reduced in groups to which 2×10⁶ and 8×10⁶ cord blood immunosuppressive cells were administered compared to a control.

Next, cord blood immunosuppressive cells were administered to a LAD ligation model, and then cardiac function was confirmed by ultrasound and compared to a control (PBS) to which cord blood immunosuppressive cells were not administered. MRI was performed using BioSpec 47/40 (Bruker, Etlingen, Germany), and dual electrocardiogram (ECG) and respiratory gating were performed. ECG signals were obtained from needle electrodes in order to obtain MR images. Signals were transmitted and received using a quadrature birdcage RF resonator (Bruker) with an internal diameter of 72 mm. ECG signals were obtained using R-waves from needle electrodes fixed on the forepaws and hindpaws for MR image acquisition. Imaging parameters: FOV=60×mm², matrix size=256×256, slice thickness=1.5 mm, number of slices=1, TR=8 ms, TE=2.8 ms, flip angle=30°, average number=6, total scan time=3 min 16s. Ejection fraction (EF) and fraction shortening (FS) were measured by M-mode tracking at the papillary muscle level.

As illustrated in FIG. 11, in a myocardial infarction mouse model, when mice to which cord blood immunosuppressive cells were administered were compared to a control to which cord blood immunosuppressive cells were not administered, the cardiac function indices of low (0.5×10⁶), mid (2.0×10⁶) and high (8.0×10⁶) groups tended to be improved compared to the control at week 2, the cardiac function indices of the low group at week 4 were reduced compared to week 2, and the mid and high groups (no difference between the two groups) exhibited a tendency to maintain the cardiac function indices.

In conclusion, administration of cord blood immunosuppressive cells (CBIC) to subjects showed effects on anti-fibrosis of myocardial infarction sites and improvement in cardiac function indices (ESV, FS, and EF) in a dose-dependent manner.

### <Example 6> Results of Confirming Viability of Cord Blood Immunosuppressive Cells

A LAD ligation model was established in order to confirm viability and dose responsiveness by intravascularly injecting cord blood immunosuppressive cells, and viability was confirmed by administering cord blood immunosuppressive cells to the model, and then comparing the LAD ligation model to a control (PBS) to which cord blood immunosuppressive cells were not administered. Furthermore, myocardial infarction was induced, and then viability was confirmed for 30 days.

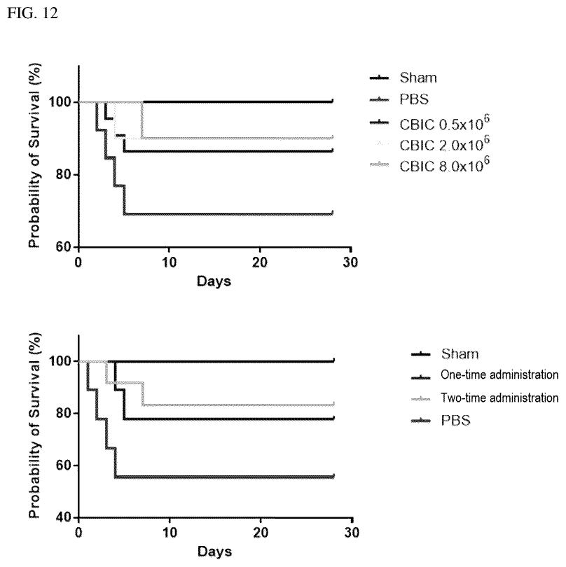

As illustrated in FIG. 12, it was confirmed that, in a myocardial infarction mouse model, the viabilities of the cord blood immunosuppressive cell injection groups in mice to which cord blood immunosuppressive cells were administered were high compared to a control to which cord blood immunosuppressive cells were not administered.

### <Example 7> Results of Confirming Migration of Cord Blood Immunosuppressive Cells (CBICs) to the Hearts of Mice with Myocardial Infarction

Cord blood immunosuppressive cells were administered to an LAD ligation model, and the migration was confirmed compared to the administration group of normal mice. gDNA was extracted after removing the lymph nodes, lungs, livers, kidneys, spleens and heart tissues of myocardial infarction and control mice. A human ALU (ALU) gene was analyzed by quantitative PCR under the conditions described in Table 1 (forward primer: 5′-ACCTGAGGTCAGGAGTTTGAGA-3′, reverse primer: 5′-ACCACGCCCGGCTAATTTT-3′).

TABLE 1

| | | | Final |
|---|---|---|---|
| component | | Volume (μl) | concentration |
| 2x Taqman universal PCR master mix | | 5 | 1x |
| Mouse liver DNA (Std), Sample | | 2 | 500 ng |
| Primer & Probe (ALU) | | 0.1 | Primer 90 nM, |
| | | | Probe 25 nM |
| DW | | 2.9 | |
| | | Temperature | Time |
| Step | Phase | (° C.) | (mm:ss) |
| 1 | Initial denaturation | 95.0 | 10:00 |
| 2 | Denaturation | 95.0 | 00:15 |
| 3 | Annealing | 56.0 | 00:30 |
| 4 | Extension | 72.0 | 00:30 |

Repeat steps 2-4 (40 cycles)

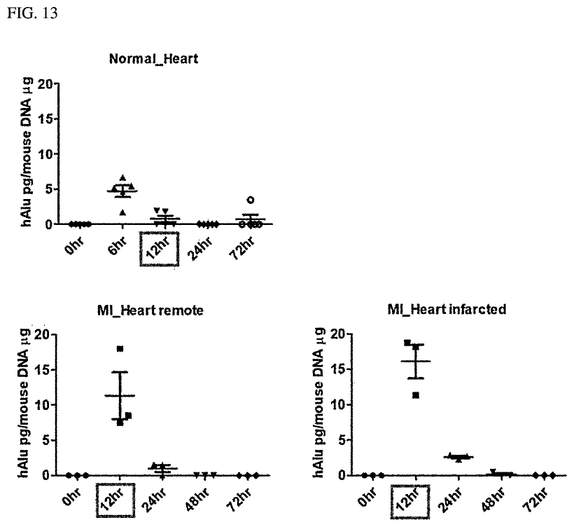

As illustrated in FIG. 13, it was confirmed that the migration of cord blood immunosuppressive cells to the heart in the myocardial infarction mouse model is 2 to 3-fold higher than that of normal mice.

The present invention can be applied to the field of preventing or treating inflammation, fibrosis, and myocardial infarction.

What is claimed is:

1. A composition for preventing or treating inflammation, fibrosis, or myocardial infarction, comprising a cord blood immunosuppressive cell.

2. The composition of claim 1, wherein the cord blood immunosuppressive cell expresses a cell phenotype comprising CD11b+, CD33+, CD14+, CD15- and HLA-DR^{LOW}.

3. The composition of claim 1, wherein the cord blood immunosuppressive cell expresses a T cell inhibitory substances comprising arginase I, inducible nitric oxide synthase (iNOS), and indoleamine 2,3-dioxygenase (IDO).

4. The composition of claim 1, wherein the cord blood immunosuppressive cell is induced by culturing CD34-positive cells isolated from human cord blood in a cell culture medium comprising a cytokine combination of GM-CSF and SCF for 2 to 7 weeks.

5. The composition of claim 4, wherein the cytokine combination of GM-CSF and SCF is comprised in the cell culture medium at a concentration ratio of 1:0.8 to 0.3.

6. The composition of claim 1, wherein the cord blood immunosuppressive cell exhibits an increase in differentiation and migration of an inflammatory suppressor cell (M2 macrophage), a decrease in differentiation and migration of a proinflammatory cell (M1 macrophage), an improvement in cardiac function (end-systolic volume (ESV), fraction shortening (FS), and ejection fraction (EF)), a decrease in myocardial infarction size or an increase in migration to the heart during myocardial infarction.

7. A method for selecting a cord blood-derived immunosuppressive cell with excellent immunosuppressive ability, the method comprising:
classifying cord blood immunosuppressive cells having a phenotype of CD33+CD11b+ and selecting a cord blood immunosuppressive cell expressing a phenotype of CD14+ using CD15 positive cells as a positive control among the cord blood immunosuppressive cells;
selecting a cord blood immunosuppressive cell having an HLA-DR^{LOW} phenotype among cord blood immunosuppressive cells having a cell phenotype of CD11b+CD33+CD14+ compared to HLA-DR-positive cells; and
co-culturing peripheral blood mononuclear cells and cord blood immunosuppressive cells under magnetic beads at a concentration of 0.125 to 2 μl/mL to confirm the proliferation ability of T cells, and
co-culturing peripheral blood mononuclear cells and an immunosuppressant, as a control, at a concentration of 0.05 to 320 ng/mL under magnetic beads at a concentration of 0.125 to 2 μl/mL to confirm the proliferation ability of T cells and compare the T cell proliferation ability of the cord blood immunosuppressive cells with that of the control,
wherein the cord blood immunosuppressive cell is induced by culturing CD34-positive cells isolated from human cord blood in a cell culture medium including a cytokine combination of GM-CSF and SCF for 2 to 7 weeks.

8. The method of claim 7, wherein the CD15 positive cells comprise any one of granulocytes; and a genetically modified cell line so as to express a CD15 gene.

9. The method of claim 7, wherein the HLA-DR-positive cells comprise any one of dendritic cells; monocytes; and a genetically modified cell line so as to express an HLA-DR gene.

10. The method of claim 7, wherein the peripheral blood mononuclear cells and cord blood immunosuppressive cells are co-cultured at a cell number ratio of 1:0.25 to 1.

11. The method of claim 7, wherein the immunosuppressant is one or more selected from the group consisting of rapamycin, cyclosporin A, tacrolimus, mycophenolic acid, azathioprine, Bredinin, silorimus, and everolimus.

12. A method for treating inflammation, fibrosis, or myocardial infarction, the method comprising: administering a therapeutically effective amount of cord blood immunosuppressive cells to a subject in need thereof.

13. The method of claim 12, wherein the cord blood immunosuppressive cell expresses a cell phenotype comprising CD11b+, CD33+, CD14+, CD15- and HLA-DR^{LOW}.

14. The method of claim 12, wherein the cord blood immunosuppressive cell expresses T cell inhibitory substances comprising arginase I, inducible nitric oxide synthase (iNOS), and indoleamine 2,3-dioxygenase (IDO).

15. The method of claim 12, wherein the cord blood immunosuppressive cell is induced by culturing CD34-positive cells isolated from human cord blood in a cell culture medium comprising a cytokine combination of GM-CSF and SCF for 2 to 7 weeks.

16. The method of claim 15, wherein the cytokine combination of GM-CSF and SCF is comprised in the cell culture medium at a concentration ratio of 1:0.8 to 0.3.

17. The method of claim 12, wherein the cord blood immunosuppressive cell exhibits an increase in differentiation and migration of an inflammatory suppressor cell (M2 macrophage), a decrease in differentiation and migration of a proinflammatory cell (M1 macrophage), an improvement in cardiac function (end-systolic volume (ESV), fraction shortening (FS), and ejection fraction (EF)), a decrease in myocardial infarction size or an increase in migration to the heart during myocardial infarction
